SEQUENCE LISTING (14 sequences)

SEQ ID NO: 1 - DNA, 15 bp, Artificial Sequence
  Description of Artificial Sequence  Antisense oligonucleotide
ggcttcactaattcc

SEQ ID NO: 2 - DNA, 15 bp, Artificial Sequence
  Description of Artificial Sequence  Antisense oligonucleotide
cgtatgacagatctg

SEQ ID NO: 3 - DNA, 18 bp, Artificial Sequence
  Description of Artificial Sequence  Antisense oligonucleotide
gccacactgctctaaccg

SEQ ID NO: 4 - DNA, 22 bp, Artificial Sequence
  Description of Artificial Sequence  Antisense oligonucleotide
ggtctctggccgctgcgcgcgg

SEQ ID NO: 5 - DNA, 20 bp, Artificial Sequence
  Description of Artificial Sequence  Antisense oligonucleotide
gcgggcgtggcacgcgcccg

SEQ ID NO: 6 - DNA, 22 bp, Artificial Sequence
  Description of Artificial Sequence  Antisense oligonucleotide
cccaagtcattcctaaggcacc

SEQ ID NO: 7 - DNA, 22 bp, Artificial Sequence
  Description of Artificial Sequence  Antisense oligonucleotide
gggagtacaggcgcaagacacc

SEQ ID NO: 8 - DNA, 23 bp, Artificial Sequence
  Description of Artificial Sequence  Antisense oligonucleotide
cgatccacctgcctcggcctccc

SEQ ID NO: 9 - DNA, 23 bp, Simian virus 40
cctcaggctatagagtagctggg

SEQ ID NO: 10 - protein, 7 aa, Simian virus 40
PKKKRKV

SEQ ID NO: 11 - protein, 6 aa, Mus musculus
ARRRRP

SEQ ID NO: 12 - protein, 10 aa, Mus musculus
EEVQRKRQKL

SEQ ID NO: 13 - protein, 9 aa, Mus musculus
EEKRKRTYE

SEQ ID NO: 14 - protein, 20 aa, Xenopus laevis
AVKRPAATKKAGQAKKKKLD

(19) United States
(12) Patent Application Publication
(10) Pub. No.: US 2002/0086840 A1
(43) Pub. Date: Jul. 4, 2002

(54) Use of Rad51 inhibitors for p53 gene therapy

(76) Inventors: David A. Zarling, Menlo Park, CA (US); Gurucharan Reddy, Fremont, CA (US)

Correspondence Address: FLEHR HOHBACH TEST, ALBRITTON & HERBERT LLP, Four Embarcarero Center, Suite 3400, San Francisco, CA, 94111-4187, US

(21) Appl. No.: 09/771,355

(22) Filed: Jan. 26, 2001

Related U.S. Application Data
(60) Provisional application No. 60/178,561, filed on Jan. 26, 2000.

(51) Int. Cl. A61K 48/00; A61K 39/395; A61K 38/17

(52) U.S. Cl. 514/44; 514/12; 424/155.1

(57) ABSTRACT

The present invention is directed to methods and compositions for inhibiting or reducing tumor cell proliferation in an individual in vivo. More specifically, a tumor cell is contacted, in vivo, with a Rad51 inhibitor, and a polynucleotide capable of expressing functional p53 protein. In a further embodiment of the present invention the tumor cell is exposed in vivo to radiation or chemotherapeutic agents (e.g., BCNU, CCNU, and DMZ, GB, cisplatin and the like). The Rad51 inhibitor may be selected from the group consisting of peptides, small molecules and Rad51 antisense molecules. The Rad51 antisense molecule and the p53 polynucleotide may be encoded on an expression vector under the control of one or more promoters, and the expression vector may then be incorporated into a viral genome, preferably an andeno or retro virus, which is then used to introduce the expression vector into the tumor cell.

### FIELD OF THE INVENTION

The present invention relates to therapeutic treatment of cancer using Rad51 inhibitors in combination with p53 gene therapy, and in further combination with chemo- or radiation therapy.

### BACKGROUND OF THE INVENTION

It is now well established that a variety of cancers are caused, at least in part, by genetic abnormalities that result in either the over expression of one or more genes, or the expression of an abnormal or mutant gene or genes. For example, in many cases, the expression of oncogenes is known to result in the development of cancer. Oncogenes are genetically altered genes whose mutated expression product somehow disrupts normal cellular function or control (Spandidos et al., J. Pathol., 157:1-10 (1989)).

Most oncogenes studied to date have been found to be activated as the result of a mutation, often a point mutation, in the expressed protein product. This altered expression product exhibits an abnormal biological funtion that takes part in the neoplastic process (Travali et al., FASEB, 4:3209-3214 (1990)). The underlying mutations can arise by various means, such as by chemical mutagenesis or ionizing radiation. A number of oncogenes and oncogene families, including ras, myc, neu, raf, erb, src, fms, jun and abl have now been identified and characterized to varying degrees (Travali et al., 1990; Bishop, Science, 235:305-311 (1987)).

During normal cell growth, it is thought that growth-promoting proto-oncogenes are counterbalanced by growth-constraining tumor suppressor genes. Several factors may contribute to an imbalance in these two forces, leading to the neoplastic state. One such factor is mutations in tumor suppressor genes (Weinberg, Science, 254:1138-1145 (1991)).

An important tumor suppressor gene is the gene encoding the cellular protein, p53, which is a 53 kD nuclear phosphoprotein that controls cell proliferation. Mutations to the p53 gene and allele loss on chromosome 17p, where this gene is located, are among the most frequent alterations identified in human malignancies. The p53 protein is highly conserved through evolution, and is expressed in most normal tissues. Wild-type p53 has been shown to be involved in control of the cell cycle (Mercer, Critic. Rev. Eukar. Gene Express., 2:251-263 (1992)), transcriptional regulation (Fields et al., Science, 249:1046-1049 (1990); Mietz et al., EMBO, 11:5013-5020 (1992)), DNA replication (Wilcock and Lane, Nature, 349:429-431 (1991); Bargonetti et al., Cell, 65:1083-1091 (1991)), and induction of apoptosis (Yonish-Rouach et al., Nature, 352:345-357 (1991); Shaw et al., PNAS, 89:44954499 (1992)).

Various mutant p53 alleles are known in which a single base substitution results in the synthesis of proteins that have quite different growth regulatory properties, and ultimately lead to malignancies (Holestein et al., Science, 253:49-53 (1991)). In fact, the p53 gene has been found to be the most frequently mutated gene in common human cancers (Hollstein et al., 1991; Weinberg, 1991). The overexpression of p53 in breast tumors, by transfection of DNA encoding wild-type p53, has been shown to restore growth suppression control (Casey et al., Oncogene, 6:1791-1797 (1991)). A similar effect has also been demostrated on transfection of wild-type, but not mutant, p53 into human lung cancer cell lines (Takahasi et al., Cancer Res., 52:2340-2342 (1992)). The p53 appears dominant over the mutant gene and will select against proliferation when transfected into cells with the mutant gene. Normal expression of the tranfected p53 does not affect the growth of cells with endogenous p53. Thus, such constructs might be taken up by normal cells without adverse effects.

Gene delivery systems applicable to gene therapy for tumor suppression are currently available. Basic transfection methods, as just described, exist in which DNA containing the gene of interest is introduced into cells non-biologically, for example, by permeabilizing the cell membrane physically or chemically. This approach is most applicable to cells that can be temporarily removed and can tolerate the cytotoxicity of the treatment, e.g., lymphocytes. Liposomes or protein conjugates formed with certain lipids and amphophilic peptides can be used for in vivo transfection.

Virus-based gene transfer vehicles is another method of transfecting DNA into cells. This approach capitalizes on the natural ability of viruses to enter cells carrying their genetic material with them. A variety of virus based vehicles can be used, such as adeno- and retro-viruses. For example, U.S. Pat. Nos. 6,069,134, 6,143,290, and 5,747,469 describe the use of human adenoviruses to transfer and express a wild-type p53 gene into cancerous cells. More specifically, a replication-defective, helper-independent andenovirus that expresses wild-type p53 under the control of the human cytomegalovirus promoter was used, in vivo, to restore p53 mediated growth suppression of lung cancer.

Thus, in vivo p53 gene therapy has been demonstrated as a therapeutically effective means of suppressing or inhibiting the proliferation of cancer cells. Additionally, other cancer fighting techniques in combination with p53 gene therapy have proven more therapeutically effective than p53 gene therapy used by itself. For example, in U.S. Pat. No. 5,747,469 it was demonstrated that use of a DNA damaging agent (e.g., chemotherapeutic drugs) in combination with restoring or enhancing cellular p53 activity by gene therapy resulted in better therapeutic effect than treatment by the agent or p53 gene therapy alone. WO 99/46371 describes introducing adenoviral vectors having a proapoptotic gene (e.g., Bax, Bak, Bim and Bad) under the control of a first promoter, and a p53 gene under the control of an Internal Ribosomal Entry Site or a second promoter. The expression of both the proapoptotic and p53 proteins in combination increased the therapeutic effect on tumors in vivo over the use of p53 gene therapy used by itself.

Rad51 protein is important for the repair of double-strand breaks in damaged cells. In S. cerevisiae, genes with homology to RecA include Rad51, Rad57 and Dmcl. Rad51 is a member of the Rad52 epistasis group, which includes Rad50, Rad51, Rad52, Rad54, Rad55 and Rad57. All these genes were initially identified as being defective in the repair of damaged DNA caused by ionizing radiation and dysfunctional mutants in these genes were subsequently shown to be deficient in both genetic recombination and the recombinational repair of DNA lesions (Yeast Genetics: Fundamental and Applied Aspects, J. F. T. Spencer and A. R. W. Smith, Eds. (New-York: Springer-Verlag):109-137 (1983); The Molecular Biology of the Yeast Saccaromyces Cerevisiae: Life Cycle and Inheritance, J. N. Strathern, E. W. Jones and J. M. Broach Eds. (Cold Spring Harbor Laboratory Press):371-414 (1981); Investigating the Genetic Control of Biochemical Events in Meiotic Recombination, P. B. Moens, Ed. (New York: Academic Press):157-210 (1987)). Recent experiments found that homozygous knock-outs of Rad51 in chicken B cells are extremely sensitive to radiation, accumulate double-stranded DNA breaks, and undergo programmed cell death (Sonoda, et al., EMBO 17:598-608 (1998)).

Although Rad51 RNA transcripts and protein are present in all cell types, the highest transcript levels are in tissue active in recombination, including spleen, thymus, ovary and testis (Morita, et al., Proc. Natl. Acad. Sci. USA 90:6577-6580 (1993)). For example, Rad51 is specifically induced in murine B cell nuclei undergoing Ig class switch recombination (Li, et al., Proc. Natl. Acad. Sci. USA 93:10222-10227 (1996)), Rad51 is enriched in the synaptonemal complexes which join paired homologous chromosomes in spermatocytes undergoing meiosis (Haaf, et al., Proc. Natl. Acad. Sci. USA 92:2298-2302 (1995); Ashley, et al., Chromosoma 104:19-28 (1995); Plug, et al., Proc. Natl. Acad. Sci. USA 93:5920-5924 (1996)), and Rad51 nuclear localization changes dramatically in response to DNA damage in cultured cell lines, when multiple discreet foci are re-distributed in the nucleus and stain vividly with anti-Rad51 antibodies (Haaf, et al., Proc. Natl. Acad. Sci. USA 92:2298-2302 (1995)).

Targeted disruption of Rad51 leads to an embryonic lethal phenotype in mouse and the dying embryo cells are very sensitive to radiation (Tsuzuki, et al., Proc. Natl. Acad. Sci. USA 93:6236-6240 (1996); Lim & Hasty, Mol. Cell. Biol. 16:7133-7143 (1996)). Attempts to generate viable homozygous Rad51^{-/-} embryonic stem cells have not been successful. These results show that Rad51 plays an essential role in cell proliferation, a surprise in view of the viability of S. cerevisiae carrying Rad51 deletions. It is also interesting that Rad51 is associated with RNA polymerase II transcription complexes (Maldonado, et al., Nature 381:86-89 (1996)). Although the specificity and functional nature of these interactions are not clear, these observations taken together point to a pleiotropic role for human Rad51 in DNA metabolism (repair, recombination, transcription), and maintenance of genomic integrity.

Human Rad51 protein interacts directly with wild type p53 protein, and the regions necessary for this interaction have been mapped (Sturzbecher et, al., EMBO 15:1992-2002 (1996); Buchhop, et al., Nucleic Acids Res 25:3868-3874 (1997)). Rad51 interacts with two different regions of p53 (amino acids 94-160 and 264-315), and p53 interacts with the region between amino acids 125 and 220 of Rad51. This latter region is necessary for homo-oligomerization of Rad51. Therefore, p53 may inhibit Rad51 activity by blocking the formation of active Rad51 oligomers. Furthermore, p53 inhibits Rad51 ATPase and DNA strand exchange activities. Interestingly, p53 mutants often found in cancer cells, are reported to bind Rad51 less efficiently than wild type 53 and fail to inhibit its biochemical activities. Taken together, known interactions between Rad51 and p53 suggest that (1) in normal cells p53 interacts with and downregulates Rad51, and (2) in tumor cells with p53 mutations, unregulated Rad51 could possibly lead to uncontrolled recombination, genetic instability, and radiation resistance by upregulating DNA recombination and DNA repair (Sturzbecher, et al., EMBO 15:1992-2002 (1996); Ohnishi, et al., Biochem. Biophys. Res. Comm. 245:319-324 (1998)).

Rad51 also interacts with BRCA1 and BRCA2 (Scully, et al., Cell 88:265-275 (1997); Sharan, et al., Nature 386:804-810 (1997)). Inherited mutations in BRCA1 cause familial breast and ovarian cancer, and inherited mutations in BRCA2 case familial breast cancer (Wooster, et al., Science 265:2088-2090 (1994); Smith, et al., Nature Genet. 2:128-131 (1992); Easton, et al., Am. J. Hum. Genet. 52:678-701 (1993); Gayther, et al., Nature Genet. 15:103-105 (1997)). Sharan, et al., J. Nature. 386:804-810 (1997) showed that BRCA2 binds to Rad51, and that mouse BRCA2 knockouts are both early embryonic lethal and hypersensitive to radiation, similar to Rad51 knockout mice. Furthermore, certain BRCA2 peptides bind Rad51 and inhibit cell growth. Scully, et al., Cell 88:265-275 (1997) showed that BRCA1 binds to Rad51 and co-localizes with it in synaptonemal complexes.

Recently, several human members of the Rad51 family of related genes have been identified, including Rad51 B (Albala, et al., Genomics 46:476-479 (1997)), Rad51C (Dosanjh, et al., Nucleic Acids Res 26:1179-1184 (1998)), Rad51 D (Pittman, et al., Genomics 49:103-111 (1998)), XRCC2 (Cartwright et al., Nucleic Acids Res 26:3084-3089-793 (1998)) and XRCC3 (Liu, et al., Mol Cell 1:783 (1998)). While these genes are homologous to human Rad51, it is also possible that they are related to certain other members of the Rad52 epistasis group such as Rad55 and Rad57. The chromosomal locations of all these genes have been mapped. XRCC2 maps to chromosome 7q36.1, a region associated with radiation resistance in human glial tumors.

Given that p53 gene therapy, alone or in combination with other therapies, is an effective tool to treat cancer additional therapeutic compositions that serve to augment or complement p53 gene therapy will improve the currently available cancer therapy regimens.

### SUMMARY OF THE INVENTION

The present invention is directed to methods and compositions for inhibiting or reducing tumor cell proliferation in an individual in vivo. More specifically, a tumor cell is contacted, in vivo, with a Rad51 inhibitor, and a polynucleotide capable of expressing functional p53 protein. In a further embodiment of the present invention the tumor cell is exposed in vivo to radiation or chemotherapeutic agents (e.g., BCNU, CCNU, and DMZ, GB, cisplatin and the like). The Rad51 inhibitor may be selected from the group consisting of peptides, small molecules and Rad51 antisense molecules. The Rad51 antisense molecule and the p53 polynucleotide may be encoded on an expression vector under the control of one or more promoters, and the expression vector may then be incorporated into a viral genome, preferably an andeno or retro virus, which is then used to introduce the expression vector into the tumor cell.

### DETAILED DESCRIPTION OF THE PREFERRED EMBODIMENTS

The present invention provides methods and compositions for treating disease states requiring reduction or inhibition of cellular proliferation. In a preferred embodiment, the disease state is typified by aberrant Rad51 activity, and aberrant p53 activity. As will be appreciated by those in the art, a disease state means either that an individual has the disease, or is at risk to develop the disease.

As described in co-pending applications U.S. Ser. Nos. 09/260,624, 09/454,495, 09/620,414, and 09/637,313 reducing or inhibiting Rad51 activity in a tumor cell, or in any diseased cell having aberrant Rad51 activity, results in an increased incidence of tumor cell death, and, therefore, a reduction or inhibition of tumor cell proliferation. In addition, as described in U.S. Pat. Nos. 6,134,290, 6,069,134, and 5,747,469 and PCT publication WO 99/46371 (for example), introduction of a vector to express functional p53 protein in a tumor cell, or in any cell having aberrant functional p53 activity, also results in an increased incidence of tumor cell death, and, therefore, a reduction or inhibition of tumor cell proliferation. All of these references are incorporated herein, in their entirety, by reference.

It is known that p53 is invlolved with controlling the cell cycle transcriptional regulation, DNA replication, and mediation of apoptosis. Without being bound by theory, it is believed that the mechanism of action in p53 gene therapy involves restoring functional p53 protein in cells significantly deficient in or lacking the same, thereby inducing the cells into apoptosis. It is also known that Rad51 protein is involved in repairing damaged DNA. Without being bound by theory, it is believed that in diseased cells unregulated Rad51 leads to uncontrolled recombination, genetic instability and radiation resistance by upregulating DNA recombination and DNA repair, thereby permitting these diseased cells to proliferate. Again without being bound by theory, in diseased cells with aberrant Rad51 activity and without functional p53 protein it is believed that inhibiting the activity of Rad51 and increasing the activity of functional p53 protein increase the ability of the diseased cell to undergo the desired apoptotic cycle, thereby reducing or eliminating proliferation of the diseased cell.

Thus, the present invention is directed to combining inhibition of Rad51 (hereinafter “Rad51 inhibition therapy”), and increasing functional p53 (hereinafter “p53 gene therapy”) in diseased cells to achieve a greater therapeutic effect than either technique used alone. Additionally, the present invention is directed to using the combination of Rad51 inhibition therapy and p53 gene therapy in further combination with DNA mutagenisis therapy (e.g., chemo or radiation therapies) to achieve a greater therapeutic effect than Rad51 inhibition therapy combined with p53 gene therapy. In particular, described herein are compositions and methods for inhibiting or reducing tumor cell proliferation by inhibiting Rad51 activity with a Rad51 inhibitor in combination with a p53 gene therapy. In an alternative embodiment, DNA mutagenisis therapy (e.g., chemo- and radiation therapies) may be further combined with Rad51 inhibition therapy and p53 gene therapy to inhibit or reduce the tumor cell proliferation. The methods of the present invention include both in vitro and in vivo applications, preferably in vivo.

Inhibition of Rad51 biological or biochemical activity as used herein can be measured from Rad51 activities selected from the group consisting of DNA dependent ATPase activity, formation of Rad51 foci, nucleic acid strand exchange, DNA binding, nucleoprotein filament formation, DNA pairing and DNA repair. DNA repair and recombination are generally considered biological activities of Rad51. DNA repair can be double stranded break repair, single stranded annealing or post replication recombinational repair.

A Rad51 inhibitor or an agent or composition having Rad51 inhibitory activity is defined herein as an agent or composition that inhibits the expression or translation of a Rad51 nucleic acid or the biological activity of a Rad51 peptide by at least 30%, more preferably 40%, more preferably 50%, more preferably 70%, more preferably 90%, and most preferably by at least 95%. In one embodiment herein, a Rad51 inhibitor inhibits expression or translation of a Rad51 nucleic acid or the activity of a Rad51 protein by 100%. In alternative embodiments, inhibition of Rad51 activity is defined as any detectable decrease in Rad51 activity compared to a control not comprising the Rad51 inhibitor. The Rad51 inhibitor can inhibit Rad51 directly or indirectly, preferably directly by interacting with at least a portion of the Rad51 nucleic acid, Rad51 mRNA, or Rad51 protein. or protein. Additionally, the inhibitors herein can be utilized individually or in combination with each other. It is understood that Rad51 inhibitors may bind to Rad51, but exclude agents which generally activate Rad51, such as DNA to which Rad51 normally binds in the process of recombinational activity, ATP, and the like.

In an alternative embodiment, Rad51 inhibitors include inhibitors of Rad51 homologues, such as RecA. Thus, in this embodiment, Rad51 as used herein refers to Rad51 and its homologues, preferably human homologues. In an alternative embodiment, Rad51 excludes non-mammalian homologues. Rad51 homologues include RecA and Rad51 homologues in yeast and in mammals. Genes homologous to E. coli RecA and yeast Rad51 have been isolated from all groups of eukaryotes, including mammals. Morita, et al., PNAS USA 90:6577-6580 (1993); Shinohara, et al., Nature Genet. 4:239-243 (1993); Heyer, Experentia, 50:223-233 (1994); Maeshima, et al., Gene 160:195-200 (1995). Human Rad51 homologues include Rad51B, Rad51C, Rad51D, XRCC2 and XRCC3. Albala, et al., Genomics 46:476-479 (1997); Dosanjh, et al., Nucleic Acids Res 26:1179(1998); Pittman, et al., Genomics 49:103-11 (1998); Cartwright, et al., Nucleic Acids Res 26:3084-3089 (1998); Liu, et al., Mol Cell 1:783-793 (1998). In preferred embodiments, Rad51 inhibitors provided herein were not previously known to inhibit RecA or other Rad51 homologues, and were not known to induce sensitization of cells to radiation. In one embodiment, Rad51 as used herein excludes homologues thereof.

Rad51 inhibitors are preferably selected from the group consisting of small molecules, Rad51 antisense molecules, and pepetides.

In a preferred embodiment, the Rad51 inhibitor is a small molecule, which is preferably 4 kilodaltons (kD) or less, or alternatively the small molecule is less than 3 kD, 2 kD, 1 kD, 0.8 kD, 0.5 kD, 0.3 kD, 0.2 kD or 0.1 kD.

The small molecule Rad51 inhibitor may be either organic or inorganic, but is preferably organic. In a preferred embodiment, the small molecule has functional groups necessary for structural interaction with proteins, particularly hydrogen bonding, and typically will include at least an amine, carbonyl, hydroxyl or carboxyl group, preferably at least two of the functional chemical groups. The small molecule Rad51 inhibitor may comprise cyclical carbon or heterocyclic structures and/or aromatic or polyaromatic structures substituted with one or more chemical functional groups. Additionally, as further discussed below, small molecules may comprise nucleotides, nucleosides, and analogues thereof. Nucleotides as used herein refer to XYP, wherein X can be U, T, G, C or A (uracil, thymine, guanine, cytosine or adenine, respectively), Y can be M, D or T (mono, di or tri, respectively), and P is phosphorous. In an alternative embodiment, nucleotides can include xathanine, hypoxathanine, isocytosine, isoguanine, etc. Analogues as used herein includes derivatives of and chemically modified nucleotides and nucleosides. In one embodiment, methyl methanesulfonate is excluded from the group of small molecules. In preferred embodiment ADP is excluded from the group of small molecules.

In an alternative embodiment, the small molecule Rad51 inhibitor is a nucleotide analogue. In a preferred embodiment, the nucleotide analogue is a nucleotide diphosphate complexed with aluminum fluoride. In one embodiment, the nucleotide analogue is selected from the group consisting of ADP.Al F4, GDP.Al F4, CDP.Al F4, UDP.Al F4 and TDP.A1F4. Alternatively, the nucleotide analogue is a non-hydrolyzable nucleotide. In a preferred embodiment, the nucleotide analogue is selected from the group consisting of ATPγS, GTPγS, UTPγS, CTPγS, TTPγS, ADPγS, GDPγS, UDPγS, CDPγS, TDPγS, AMPγS, GMPγS, UMPγS, CMPγS, TMPγS, ATP-PNP, GTP-PNP, UTP-PNP, CTP-PNP, TTP-PNP, ADP-PNP, GDP-PNP, UDP-PNP, CDP-PNP, TDP-PNP, AMP-PNP, GMP-PNP, UMP-PNP, CMP-PNP, and TMP-PNP. In a preferred embodiment, ADPγS is excluded. In an alternative embodiment, the nucleotide analogue is selected from the group consisting of halogenated pyrimidines, such as 5-fluoro, 5-bromo, 5-iodo, and 5-chloro -cytidine, -uridine and -thymidine. In an alternative embodiment the halogenated pyrimidines include mono, di, and tri-phosphate derivatives, and -γS dervivatives, as will be appreciated to those skilled in the art.

In another alternative embodiment, the small molecule Rad51 inhibitor is a DNA minor groove binding drug. In a preferred embodiment, the minor groove binding drug is selected from the group consisting of distamycin, netropsin, bis-benzimidazole and actinomycin.

In a preferred embodiment of the present invention, the Rad51 inhibitor is a peptide. By “peptide” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration, preferably in the (S) or L configuration. If non-naturally occurring side chains are used, non-amino acid substituents may be used, for example to prevent or retard in vivo degradations.

The peptide Rad51 inhibitor can be naturally occurring or fragments of naturally occurring proteins. Thus, for example, cellular extracts containing proteins, or random or directed digests of proteinaceous cellular extracts may be used. Prokaryotic and eukaryotic proteins can be Rad51 inhibitors. Peptide Rad51 inhibitors may also be peptides from bacterial, fungal, viral, and mammalian sources, with the latter being preferred, and human proteins being especially preferred.

In a preferred embodiment, the peptide Rad51 inhibitors are from about 5 to about 30 amino acids, with from about 5 to about 20 amino acids being preferred, and from about 7 to about 15 being particularly preferred. The peptide Rad51 inhibitor may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids to allow the formation of all or most of the possible combinations over the length of the sequence. Preferred peptide Rad51 inhibitors include but are not limited to amino acids 94-160 and 264-315 of p53 and fragments of Rad51 antibodies.

In a preferred embodiment, the Rad51 inhibitors are nucleic acids. By “nucleic acid” or “oligonucleotide” or grammatical equivalents herein means at least two nucleotides covalently linked together. A nucleic acid of the present invention will generally contain phosphodiester bonds. However, in some cases, as outlined below, nucleic acid analogues are included that may have alternate backbones, comprising, for example, phosphoramide (Beaucage et al., Tetrahedron 49(10):1925 (1993); Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984); Letsinger et al., J. Am. Chem. Soc. 110:4470 (1988); Pauwels et al., Chemica Scripta 26:141 (1986)), phosphorothioate (Mag et al., Nucleic Acids Res. 19:1437 (1991); U.S. Pat. No. 5,644,048), phosphorodithioate (Briu et al., J. Am. Chem. Soc. 111:2321 (1989)), O-methylphosphoroamidite linkages (Eckstein, Oligonucleotides and Analogues: a Practical Approach (Oxford University Press)), and peptide nucleic acid backbones and linkages (Egholm, J. Am. Chem. Soc. 114:1895 (1992); Meier et al., Chem. Int. Ed. Engl. 31:1008 (1992); Nielsen, Nature 365:566 (1993); Carlsson et al., Nature 380:207 (1996)). Other analogue nucleic acids include those with positive backbones (Denpcy et al., Proc. Natl. Acad. Sci. USA 92:6097 (1995)), non-ionic backbones (U.S. Pat. Nos. 5,386,023, 5,637,684, 5,602,240, 5,216,141, 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)), and non-ribose backbones (U.S. Pat. Nos. 5,235,033, 5,034,506; Chapters 6 and 7, ASC Symposium Series 580, “Carbohydrate Modifications in Antisense Research”, Ed. Y. S. Sanghui and P. Dan Cook). Nucleic acids containing one or more carbocyclic sugars are also included within the definition of nucleic acids (Jenkins et al., Chem. Soc. Rev. pp169-176 (1995)). Several nucleic acid analogues are described in Rawls, C & E News p.35 (Jun. 2, 1997). All of these references are incorporated herein, in their entirety, by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs including PNA can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid (where the nucleic acid contains any combination of deoxyribo- and ribo-nucleotides). The nucleic acid may contain any combination of bases, including without limitation uracil, adenine, thymine, cytosine, guanine, inosine, xathanine hypoxathanine, isocytosine, and isoguanine.

The nucleic acids herein, including antisense nucleic acids, as further described below, are recombinant nucleic acids. A recombinant nucleic acid is distinguished from naturally occurring nucleic acid by at least one or more characteristics. For example, the nucleic acid may be isolated or purified away from some or all of the nucleic acids and compounds with which it is normally associated in its wild-type host, and thus may be substantially pure. For example, an isolated nucleic acid is unaccompanied by at least some of the material with which it is normally associated in its natural state, preferably constituting at least about 0.5%, more preferably at least about 5% by weight of the total nucleic acid in a given sample. A substantially pure nucleic acid comprises at least about 75% by weight of the total nucleic acid, with at least about 80% being preferred, and at least about 90% being particularly preferred. Alternatively, the recombinant molecule could be made synthetically, i.e., by a polymerase chain reaction, and does not need to have been expressed to be formed. The definition includes the production of a nucleic acid from one organism in a different organism or host cell.

As generally for proteins, nucleic acid Rad51 inhibitors may be naturally occuring nucleic acids, random nucleic acids, or “biased” random nucleic acids. For example, digests of prokaryotic or eukaryotic genomes may be used as is outlined above for proteins.

In an alternative embodiment the nucleic acid Rad51 inhibitor is a Rad51 antisense molecule. Preferably the Rad51 antisense molecule is at least about 10 nucleotides in length, more preferably at least 12, and most preferably at least 15 nucleotides in length. In an alternative embodiment the Rad51 antisense molecule is a morpholino based antisense molecule. Nasevicius, A. and Eker, S., Nature Genetics, 26(2):216-220 (2000); Heasman et al. Developmental Biology, 222:124-134 (2000). The skilled artisan understands that the length can extend from 10 nucleotides or more to any length which still allows binding to the Rad51 mRNA. Preferably, the length is about 30 nucleotides, more preferably about 25 nucleotides, and most preferably about 12 to 25 nucleotides in length.

The Rad51 antisense molecules hybridize under normal intracellular conditions to the target nucleic acid to inhibit Rad51 expression or translation. In an alternative embodiment an anti-gene may be used. The target nucleic acid is either DNA or RNA. In one embodiment, the antisense molecules bind to regulatory sequences for Rad51. Alternatively, the antisense molecules bind to 5′ or 3′ untranslated regions directly adjacent to the coding region of the Rad51 gene. Preferably, the antisense molecules bind to the nucleic acid within 1000 nucleotides of the coding region, either upstream from the start or downstream from the stop codon. In a preferred embodiment, the antisense molecules bind within the coding region of the Rad51 gene. More preferably, the Rad51 antisense molecule is selected from the group consisting of AS4, AS5, AS6, AS7, AS8 and AS9 as indicated in FIG. 1 and Table 1 below. Table 1 includes the recitation of “R51” before the same corresponding antisense, but “AS4” and “R51AS4”, for example, are used interchangeably herein. In one embodiment, the Rad51 antisense molecules are not directed to the structural gene; this embodiment is particularly preferred when the Rad51 antisense molecule is not combined with another antisense molecule. It is understood that any of the antisense molecules can be combined.

TABLE 1
Antisense Oligonucleotide Sequences
ANTISENSE IN CODING REGION

| | |
|---|---|
| R51AS1 | 5′-(P=S) GGC TTC ACT AAT TCC-3′ |
| R51AS2 | 5′-(P=S) CGT ATG ACA GAT CTG-3′ |
| R51AS3 | 5′-(P=S) GCC ACA CTG CTC TAA CCG 3′ |

ANTISENSE IN 5′ UNTRANSLATED REGION

| | |
|---|---|
| R51AS4 | 5′ (P=S) GGT CTC TGG CCG CTG CGC GCG G-3′ |
| R51AS5 | 5′ (P=S) GCG GGC GTG GCA CGC GCC CG-3′ |

ANTISENSE IN 3′ UNTRANSLATED REGION

| | |
|---|---|
| R51AS6 | 5′ (P=S) CCC AAG TCA TTC CTA AGG CAC C-3′ |
| R51AS7 | 5′ (P=S) GGG AGT ACA GGC GCA AGA CAC C-3′ |
| R51AS8 | 5′ (P=S) CGA TCC ACC TGC CTC GGC CTC CC-3′ |
| R51AS9 | 5′ (P=S) CCT CAG GCT ATA GAG TAG CTG GG-3′ |

The skilled artisan will appreciate that Rad51 inhibitors may be obtained from a wide variety of sources, including libraries of synthetic or natural compounds. Any number of techniques are available for the random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications to produce structural analogs.

Introduction of functional p53 protein into tumor cells lacking the same has been shown to reduce or inhibit the proliferation of the diseased cells. Casey et al., 1991; Takahasi et al. 1992. p53 gene therapy has been used to inhibit or reduce the proliferation of diseased cells deficient in functional p53 protein by introducing and expressing polynucleotides encoding functional p53 protein in the diseased cells. Id. For example, U.S. Pat. No. 6,143,290 describes making an expression vector having a polynucleotide that encodes functional p53 protein, a promoter to control the expression of the polynucleotide, and a polyadenylation signal. The expression vector is then incorporated into a replication-deficient adenovirus, preferably replacing the E1 region. The recombinant replication-deficient adenovirus is then used to infect diseased cells with deficient functional p53 protein. The infection results in the expression of functional p53 protein in the diseased cells, thereby reducing or inhibiting the proliferation of the diseased cells.

In a preferred embodiment of the present invention, the expression vector has a first polynucleotide that encodes functional p53 protein, a second polynucleotide encoding a Rad51 antisense molecule, a first promoter for the p53 polynucleotide, a second promoter for the Rad51 antisense molecule, and a polyadenylation signal. In one embodiment the first and second promoters may be the same. The expression vector is then incorporated into a replication-deficient adenovirus, or other suitable transfection vehicle, and introduced into a diseased cell. The infection results in the expression of functional p53 protein in the diseased cells and the transcription of Rad51 antisense molecule. The combination of the functional p53 protein and the inhibition of Rad51 activity results in the reduction and inhibition of diseased cell proliferation.

Alternatively, the Rad51 antisense (or other Rad51 inhibitor as discussed herein), and functional p53 protein are delivered to a diseased cell, thereby eliminating the need for introducing an expression vector to the diseased cell, as described above. As will be appreciated by the skilled artisan, any combination of the the techniques for delivering Rad51 inhibitor, and functional p53 protein to a diseased cell may be used.

As described above and in addition to Rad51 antisense molecules, the Rad51 inhibitor used in combination with p53 gene therapy may be selected from the group consisting of small molecules (including nucleotides and analogues thereof, as described above), or peptides

Administration of the Rad51 inhibitor may occur in a number of ways, and may be simultaneous with, before or after p53 gene therapy has occurred. Numerous techniques are available for introducing a Rad51 inhibitor into cells. The addition of the Rad51 inhibitor to a cell will be done as is known in the art for other inhibitors. The techniques vary depending upon whether the inhibitor is transferred into cultured cells in vitro, or in vivo in the cells of the intended host, as will be appreciated by the skilled artisan. For example and without limitation, techniques suitable for the transfer of inhibitors into mammalian cells in vitro include the use of liposomes, electroporation, microinjection, cell fusion, DEAE-dextran, and the calcium phosphate precipitation method. The currently preferred in vivo transfer techniques include transfection with viral (typically retroviral or adenoviral) vectors and viral coat protein-liposome mediated transfection (Dzau et al., Trends in Biotechnology 11:205-210 (1993)). Liposomes, modified electroporation, chemical treatment or piezo injection techniques are particularly preferred. In some situations it is desirable to couple the Rad51 inhibitor with an agent that targets the diseased cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. USA 87:3410-3414 (1990).

Additionally, and not by way of limitation, Rad51 inhibitor delivery may include the use of nuclear localization signal (NLS). This is especially preferred when the Rad51 inhibitor is a peptide. NLSs are generally short, positively charged (basic) domains that serve to direct the entire protein in which they occur to the cell's nucleus. Numerous NLS amino acid sequences have been reported including single basic NLSs, such as the SV40 (monkey virus) large T Antigen (Pro Lys Lys Lys Arg Lys Val) (Kalderon (1984), et al., Cell 39:499-509), the human retinoic acid receptor-B nuclear localization signal (ARRRRP), NFγB p50 (EEVQRKRQKL) (Ghosh et al., Cell 62:1019 (1990)), NFγB p65 (EEKRKRTYE) (Nolan et al., Cell 64:961 (1991)), and others (see for example Boulikas, J. Cell. Biochem. 55(1):32-58 (1994)). All of these references are incorporated herein in their entirety by reference. Double basic NLSs are exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp) (Dingwall, et al., Cell 30:449-458, (1982); Dingwall, et al., J. Cell Biol., 107:641-849; (1988)). Numerous localization studies have demonstrated that NLSs incorporated in synthetic peptides or grafted onto reporter proteins or other molecules not normally targeted to the cell nucleus cause these molecules to be concentrated in the nucleus. See, e.g., Dingwall and Laskey, Ann, Rev. Cell Biol. 2:367-390, (1986); Bonnerot, et al., Proc. Natl. Acad. Sci. USA 84:6795-6799, (1987); Galileo, et al., Proc. Natl. Acad. Sci. USA 87:458-462, (1990).

Rad51 inhibitors and p53 expression vectors (including replication-deficient adenoviruses comprising the p53 expression vector) may be administered in a variety of ways, orally, systemically, topically, parenterally (e.g., subcutaneously, intraperitoneally, and intravascularly). In one embodiment, the inhibitors are applied directly to the site of a tumor (or a site of a removed tumor) intra-operatively, or by other means of directly accessing the tumor (e.g., aspirator for treating lung tumors, catheters etc.). Depending upon the manner of administration, the Rad51 inhibitor and p53 expression vectors may be formulated in a variety of ways. The concentration of therapeutically active compound in the formulation may vary from about 0.1-100 wt. %. Generally, a therapeutic amount for the need is used, for example, to achieve reduction or inhibition of cellular proliferation, and/or induction of apoptosis within the diseased cells.

The Rad51 inhibitors and p53 expression vector can be combined in admixture with a pharmaceutically or physiologically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

The pharmaceutical compositions can be prepared in various forms, such as granules, aerosols, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York pp. 42-96 (1989).

Disease states which can be treated by the methods and compositions provided herein include, but are not limited to hyperproliferative disorders. More particularly and without limitation, the methods can be used to treat cancer (further discussed below), premature aging, autoimmune disease, arthritis, graft rejection, inflammatory bowel disease, proliferation induced after medical procedures (such as but not limited to surgery and angioplasty). Thus, in one embodiment, the invention herein includes application to cells or individuals afflicted or impending affliction with any one of these disorders. In a preferred embodiment the targeted cells or the cells of the targeted tissue are deficient in functional p53 protein, and have aberrant Rad51 activity.

The compositions and methods provided herein are particularly useful for the treatment of cancer including solid tumors such as skin, breast, brain, cervical carcinomas, pancreas, testicular carcinomas, etc. More particularly, cancers that may be treated by the compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastom, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma [embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myclodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

The individual, or patient, is generally a human subject, although as will be appreciated by those in the art, the patient may be animal as well. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient. In a preferred embodiment, the individual requires inhibition of cell proliferation. More preferably, the individual has cancer or a hyperproliferative cell condition.

The compositions provided herein may be administered in a physiologically acceptable carrier to a host, as previously described. Preferred methods of administration include systemic or direct administration to a tumor cavity or cerebrospinal fluid (CSF).

In an alternative embodiment of the present invention, Rad51 inhibitors, p53 gene therapy, and mutagenesis treatment (for example and without limitation, alkylating agents, DNA cross-linkers (intra and inter strand), cisplatin, and radiation) are used to reduce or inhibit cellular proliferation of diseased cells, preferably tumor cells. It is believed that this combination, in accordance with the present invention, provides a better therapeutic effect than any of the treatments used alone or in any combination. The skilled artisan will recognize that the particular circumstances will dictate whether using mutagenis treatment is advisable. It has been shown that Rad51 inhibitors increased the sensitivity of diseased cells to radiation treatment (also called sensitization or hypersensitization). U.S. Ser. Nos. 09/260,624, 09/454,495. Additionally, it has been shown that p53 gene therapy also increased the sensitivity of diseased cells to radiation treatment. U.S. Pat. Nos. 5,747,469; 6,069,134. Sensitization, as used herein, is measured by the tolerance of a cell to radiation or alkylating agents. For example, sensitization (measured by toxicity for example), occurs if toxicity is increased by at least 20%, more preferably at least 40%, more preferably at least 60%, more preferably at least 80%, and most preferably by 100% to 200% or more.

For the purposes of the present application the term ionizing radiation shall mean all forms of radiation (including but not limited to alpha, beta, gamma and ultra violet radiation), that are capable of directly or indirectly damaging the genetic material of a cell or virus. The term irradiation shall mean the exposure of a sample of interest to ionizing radiation, and term radiosensitive shall refer to cells or individuals which display unusual adverse consequences after receiving moderate, or medically acceptable (i.e., nonlethal diagnostic or therapeutic doses), exposure to ionizing irradiation. Alkylating agents include BCNU, CCNU temozolomide (TMZ) and O⁶-benzylguanine (BG). Additionally, radiation sensitizers (e.g., xanthine, xanthine derivatives, including caffeine, and hologenated pyrimidine nucleotides, as defined above) can be applied in any sequence with the Rad51 inhibitor and p53 gene therapy.

In one embodiment herein, the Rad51 inhibitors provided herein are administered to prolong the survival time of an individual suffering from a disease state requiring the inhibition of the proliferation of cells. In a preferred embodiment, the individual is further administered radiation or an alkylating agent.

In yet another aspect of the invention, a fragment of Rad51 is provided wherein said fragment consists essentially of a binding site for a small molecule, wherein said small molecule regulates the biological or biochemical activity of Rad51. Preferably, the regulation is inhibitory. In one embodiment, the binding site is the binding site for p53, or other tumor suppressor protein. In an alternative embodiment embodiment the binding site is the binding site for nucleotides or nucleosides.

Generally, the binding site is identified by combining the inhibitor with fragments of Rad51. In one embodiment, the fragments are from between amino acids 125 and 220. In one embodiment, Rad51 125-220 is fragmented to fragments of 5-25 amino acids and then tested separately or in random recombinations to determine the binding site by standard binding techniques.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are specifically incorporated by reference in their entirety.

What is claimed is:

1. A method for inhibiting or reducing tumor cell proliferation in an individual in vivo comprising:
contacting a tumor cell in vivo with a Rad51 inhibitor, and a polynucleotide capable of expressing functional p53 protein.

2. The method according to claim 1 further comprising:
exposing said tumor cell in vivo to radiation or chemo therapies.

3. The method according to claim 1 or 2, wherein said Rad51 inhibitor is selected from the group consisting of Rad51 antisense molecules, small molecules, peptides or antibodies.

4. The method according to claim 1 or 2, wherein said Rad51 inhibitor is a Rad51 antisense molecule.

5. The method according to claim 4, wherein the step of contacting said antisense molecule further comprises:
introducing to said tumor cell in vivo an expression vector comprising a eukaryotic functional promoter and a polynucleotide sequence encoding a Rad51 antisense molecule, wherein said polynucleotide sequence is under transcriptional control of said eukaryotic functional promoter.

6. The method according to claim 5, wherein said expression vector is an adenoviral or retroviral expression vector.

7. The method according to claim 4, wherein said antisense molecule is introduced locally to said tumor cell.

8. The method according to claim 4 further comprising introducing to said tumor cell in vivo an expression vector comprising:
(i) a first polynucleotide sequence encoding a Rad51 antisense molecule; and
(ii) a second polynucleotide sequence encoding said functional p53 protein,
wherein said first and second polynucleotides are operably linked to one or more promoter sequences which are functional in said tumor cell to produce said Rad51 antisense molecule and said functional p53 protein

9. The method according to claim 4, wherein said Rad51 antisense molecule is selected from the group consisting of AS4, AS5, AS6, AS7, AS8 and AS9.

10. The method according to claim 1 or 2, wherein said Rad51 inhibitor is a small molecule.

11. The method according to claim 10, wherein said small molecule is introduced locally to said tumor cell.

12. The method according to claim 10, wherein said small molecule is selected from the group consisting of nucleotide diphosphate, a nucleotide analogue, a DNA minor groove binding drug, a xanthine, a xanthine derivative, and halogenated pyrimidines.

13. The method according to claim 10, wherein said inhibitor is a nucleotide analogue selected from the group consisting of a nucleotide diphosphate complexed with aluminum fluoride and a non-hydrolyzable nucleotide.

14. The method according to claim 13, wherein said nucleotide diphosphate complexed with aluminum fluoride is selected from the group consisting of ADP.AlF4, GDP.AlF4, CDP.AlF4, UDP.AlF4 and TDP.AlF4.

15. The method according to claim 14, wherein said non-hydrolyzable nucleotide is selected from the group consisting of ATPγS, GTPγS, UTPγS, CTPγS, TTPγS, ADPγS, GDPγS, UDPγS, CDPγS, TDPγS, AMPγS, GMPγS, UMPγS, CMPγS, TMPγS, ATP-PNP, GTP-PNP, UTP-PNP, CTP-PNP, TTP-PNP, ADP-PNP, GDP-PNP, UDP-PNP, CDP-PNP, TDP-PNP, AMP-PNP, GMP-PNP, UMP-PNP, CMP-PNP, TMP-PNP, and hologenated pyrimidines.

16. The method according to claim 1 or 2, wherein said Rad51 inhibitor is a peptide.

17. The method according to claim 16, wherein said peptide is a p53 peptide having a higher affinity for Rad51 binding the p53 protein.

18. A method for sensitizing tumor cells in vivo to radiation comprising:
(a) introducing to a tumor cell in vivo a Rad51 inhibitor; and
(b) introducing to said tumor cell in vivo wild-type p53 protein.
